(19) United States
(12) Patent Application Publication
(10) Pub. No.: US 2019/0328013 A1
(43) Pub. Date: Oct. 31, 2019

(54) Formulation and Method of Use

(71) Applicant: ATP Institute PTY LTD, Springwood, Queensland (AU)

(72) Inventor: Matthew Legge, Springwood, Queensland (AU)

(73) Assignee: ATP Institute PTY LTD, Springwood, Queensland (AU)

(21) Appl. No.: 15/998,840

(22) PCT Filed: Feb. 16, 2017
(86) PCT No.: PCT/AU2017/050136

§ 371 Date: Aug. 16, 2018

(30) Foreign Application Priority Data
Feb. 16, 2016 (AU) 2016900527

(51) Int. Cl. A23L 27/12; A23L 33/12; A61K 35/60; A61K 35/57; A61K 35/612; A61K 36/534; A61K 36/752; A61K 36/28; A61K 36/889; A61K 9/00

(52) U.S. Cl. CPC A23L 27/13; A23L 33/12; A61K 35/60; A61K 35/57; A61K 9/0053; A61K 36/534; A61K 36/752; A61K 36/28; A61K 36/889; A61K 35/612

(57) ABSTRACT

Provided is an orally administrable formulation comprising a flavour oil that includes menthol and/or limonene and an edible oil, in which both the flavour oil and the edible oil possess anti-inflammatory properties. Methods of using the aforementioned orally administrable formulation for decreasing and/or preventing an increase in body fat and/or body weight and the prevention and/or treatment of an inflammatory disease, disorder or condition, including obesity, asthma and inflammatory bowel disease, are also provided. Also provided are methods of producing the aforementioned orally administrable formulation.

### TECHNICAL FIELD

THIS INVENTION relates to anti-inflammatory and/or anti-obesity formulations and methods of treatment and/or use thereof. The formulations are useful for a variety of cosmetic and/or therapeutic applications, such as enhancing weight loss and/or the treatment of inflammation in such conditions, including obesity, asthma and inflammatory bowel disease (IBD).

### BACKGROUND

The body may use numerous substrates, including carbohydrate, protein, fat, ketones and alcohol, as a source of fuel. To specifically support and/or encourage fat to be used as the primary fuel source there are many obstacles for the body to overcome. To this end, there exists a hierarchy that controls which fuel source is used first and this is generally governed by substrate availability, hormonal profile, stress levels, activity type and intensity, as well as signals from the sensations of taste and smell that may predict substrate availability.

Most fat burning supplements are not as effective as they could be as they may include energy substrates, such as carbohydrates, modified carbohydrates (e.g., sugar alcohols), ketones, proteins and amino acids, that may subsequently compete with fat for utilization by the user's body. By way of example, protein can be converted to glucose via gluconeogenesis and amino acids, particularly branch chain amino acids, can be ketogenic or glycogenic and compete with fat to be utilized by the body as a source of fuel. Further, with respect to carbohydrates, sweet taste receptors located in the oral cavity as well as in the pancreas and adipocytes can be activated to induce a cascade of events that prepares the body to use carbohydrate instead of fat. Additionally, the non-nutritive sweeteners typically found in such supplements can create a biochemical and hormonal profile to prepare for the influx of carbohydrate after activation of the body's sweet taste receptors, thereby further reducing the efficacy of such fat burning supplements.

Accordingly, there exists a need for improved formulations or supplements that in addition to supporting exercise performance and/or maintaining lean muscle mass, further promote the use of the body's fat stores as an energy source before, during and/or after exercise.

Inflammation is a non-specific reaction mounted by the immune system in response to a perceived injury or threat. It is an innate defensive response, distinguished from the more precisely tailored adaptive responses of the immune system. Inflammation may work cooperatively with adaptive responses of the immune system, which develop more slowly but are more precisely targeted to a harmful agent, such as a pathogen, that may be causing localised injury.

Inflammation can occur in response to many types of injury, including physical trauma, burns (e.g., from radiation, heat or corrosive materials), chemical or particulate irritants, bacterial or viral pathogens, and localized oxygen deprivation (i.e., ischemia). Inflammation includes the classic symptoms of redness, heat, swelling, and pain, and may be accompanied by decreased function of the inflamed organ or tissue.

Obesity has also been demonstrated to be a cause as well as a consequence of inflammation. In this regard, inflammation may contribute to adipogenesis, insulin resistance, fatty liver and many other causative factors of obesity. Adipocytes can also be a source of inflammatory adipokines that create a biochemical trap that may require anti-inflammatory compounds to treat. Inflammation may also be associated with autoimmune diseases and allergic reactions, such as asthma, rheumatoid arthritis and inflammatory bowel disease.

While a number of methods for treating inflammation are known, all of them have limitations, particularly with regard to broad based efficacy. Thus, there is a need for new methods for reducing, alleviating and/or preventing inflammation associated with a variety of causes.

### SUMMARY

The present invention is directed to formulations and methods of treating and/or preventing inflammation. Further, the invention is directed to formulations and methods for promoting and/or enhancing fat burning and/or weight loss.

In a broad form, the invention relates to orally administrable formulations comprising a flavour oil and an edible oil for use in the treatment of an inflammatory disease, disorder or condition, such as obesity, asthma, inflammatory bowel disease, irritable bowel syndrome, arthralgia, insulin resistance syndrome and metabolic syndrome. Additionally, formulations comprising a flavour oil and an edible oil as described herein may be useful in promoting and/or enhancing fat burning and, as such, may be effective in decreasing body fat and/or body weight in a subject.

In a first aspect, the invention provides an orally administrable formulation comprising, consisting or consisting essentially of:
(i) a flavour oil comprising menthol, limonene and/or one or more components or derivatives thereof; and
(ii) an edible oil;
   wherein both the flavour oil and the edible oil have one or more anti-inflammatory properties.

In one embodiment, the flavour oil is selected from the group consisting of peppermint oil, grapefruit oil, rosemary oil, lemon oil and any combinations thereof.

Suitably, the edible oil comprises one or more of an omega 3 fatty acid, an omega 6 fatty acid, punicic acid, an omega 7 fatty acid, an omega 9 fatty acid, and any components, variants or derivatives thereof. Preferably, the omega 3 fatty acid is selected from the group consisting of a-linolenic acid (ALA), hexadecatrienoic acid (HTA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA; clupanodonic acid), docosahexaenoic acid (DHA), tetracosapentaenoic acid, tetracosahexaenoic acid (nisinic acid) and any combination thereof. Preferably, the omega 6 fatty acid is selected from the group consisting of linoleic acid, γ-linolenic acid (GLA), calendic acid, eicosadienoic acid, dihomo-gamma-linolenic acid (DGLA), docosadienoic acid, adrenic acid, docosapentaenoic acid, tetracosatetraenoic acid, tetracosapentaenoic acid and any combination thereof. Preferably, the omega 7 fatty acid is selected from the group consisting of palmitoleic acid, vaccenic acid, paullinic acid and any combination thereof. Preferably, the omega 9 fatty acid is selected from the group consisting of oleic acid, erucic acid, elaidic acid, gondoic acid, mead acid, nervonic acid, and any combination thereof.

In one embodiment, the edible oil is selected from the group consisting of fish oil, krill oil, safflower oil, coconut oil, pomegranate seed oil, palm oil, emu oil, and any combination thereof.

Suitably, the formulation comprises from about 0.5% to about 20% of the flavour oil and from about 80% to about 99.5% of the edible oil. Preferably, the formulation comprises from about 2.5% to about 10% of the flavour oil and from about 90% to about 97.5% of the edible oil.

Suitably, the formulation comprises about 1% to about 20% peppermint oil and about 80% to about 99% safflower oil. Preferably, the formulation comprises about 5% peppermint oil and about 95% safflower oil.

Suitably, the formulation comprises about 1% to about 20% grapefruit oil, about 0.5% to about 20% peppermint oil, about 10% to about 90% safflower oil, about 10% to about 90% pomegranate seed oil and about 10% to about 90% coconut oil. Preferably, the formulation comprises about 2.5% grapefruit oil, about 7.5% peppermint oil, about 45% safflower oil, about 20% pomegranate seed oil and about 25% coconut oil.

Suitably, the formulation comprises about 0.5% to about 20% grapefruit oil and about 80% to about 99.5% krill oil. Preferably, the formulation comprises about 2.5% grapefruit oil and about 97.5% krill oil.

Suitably, the formulation comprises about 0.5% to about 20% grapefruit oil, about 0.5% to about 20% rosemary oil and about 60% to about 90% fish oil. Preferably, the formulation comprises about 2.5% grapefruit oil, about 2.5% rosemary oil and about 95% fish oil.

Suitably, the formulation comprises about 0.5% to about 20% lemon oil and about 80% to about 99.5% fish oil. Preferably, the formulation comprises about 2.5% lemon oil and about 97.5% fish oil.

Suitably, the formulation comprises about 0.5% to about 20% grapefruit oil and about 80% to about 99.5% fish oil. Preferably, the formulation comprises about 2.5% grapefruit oil and about 97.5% fish oil.

Suitably, the formulation comprises about 0.5% to about 20% grapefruit oil and about 80% to about 99.5% safflower oil. Preferably, the formulation comprises about 2.5% grapefruit oil and about 97.5% safflower oil.

In particular embodiments, the formulation is substantially free of carbohydrate, protein, dietary fibre, alcohol, fillers, and/or sweetener.

In a second aspect, the invention provides a method of producing the orally administrable formulation according to the first aspect, including the step of combining the flavour oil comprising menthol, limonene and/or one or more components or derivatives thereof; and the edible oil, wherein both the flavour oil and the edible oil have anti-inflammatory properties, to thereby produce the orally administrable formulation.

In a third aspect, the invention provides an orally administrable formulation according to the first aspect, or produced according to the method of the second aspect for use in:

(i) the therapeutic and/or prophylactic treatment of an inflammatory disease, disorder or condition; and/or,

(ii) decreasing and/or preventing an increase in body fat and/or body weight; in a subject.

In a fourth aspect, the invention provides a method of treating and/or preventing an inflammatory disease, disorder or condition in a subject, said method including the step of administering to said subject a therapeutically effective amount of the orally administrable formulation of the first aspect or produced according to the method of the second aspect, to thereby treat and/or prevent said inflammatory disease, disorder or condition in the subject.

Suitably, the inflammatory disease, disorder or condition is selected from the group consisting of Addison's disease, allergic rhinitis, Alzheimer's disease, amyotrophic lateral sclerosis (ALS), ankylosing spondylitis, asthma, atherosclerosis, auto-immunity, cancer-related inflammation, candidiasis, celiac disease, chronic bronchitis, chronic inflammatory demyelinating polyneuropathy (CIDP), chronic obstructive pulmonary disease (COPD), chronic recurrent multifocal osteomyelitis (CRMO), Crohn's disease, ulcerative colitis, dementia, demyelinating neuropathies, eczema, emphysema, glomerulonephritis, food allergy, food intolerance, Good pasture's syndrome, gouty arthritis, Graves' disease, Guillain-Barre syndrome, Hashimoto's encephalitis, Hashimoto's thyroiditis, hypertension, hypercholesterolemia, hypogammaglobulinemia, idiopathic thrombocytopenic purpura (ITP), infection, infestation, inflammatory bowel disease (IBD), insulin resistance, insulin resistance syndrome, insulin-dependent diabetes (type 1), intestinal dysbiosis, juvenile arthritis, Kawasaki syndrome, metabolic syndrome, multiple sclerosis, myasthenia gravis, non-alcoholic hepatic steatorrhoea, osteoarthritis, Parkinson's disease, polycystic ovarian syndrome, postmyocardial infarction syndrome, primary biliary cirrhosis, psoriasis, idiopathic pulmonary fibrosis, reactive arthritis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis.

In a fifth aspect, the invention provides a method of decreasing and/or preventing an increase in body fat and/or body weight in a subject, said method including the step of administering to said subject a therapeutically effective amount of the orally administrable formulation according of the first aspect or produced according to the method of the second aspect, to thereby decrease and/or prevent an increase in body fat and/or body weight in the subject.

In particular embodiments, the orally administrable formulation is administered to the subject together with a ketogenic diet.

In one embodiment, the orally administrable formulation promotes, enhances and/or prolongs ketosis in the subject.

In further embodiments, the orally administrable formulation is administered before, during and/or after an exercise.

With respect to the third, fourth and fifth aspects, the subject is suitably a human.

Throughout this specification, unless otherwise indicated, “comprise”, “comprises” and “comprising” are used inclusively rather than exclusively, so that a stated integer or group of integers may include one or more other non-stated integers or groups of integers. Conversely, the terms “consist”, “consists” and “consisting” are used exclusively, such that a stated integer or group of integers are required or mandatory, and no other integers may be present.

The phrase “consisting essentially of” indicates that a stated integer or group of integers are required or mandatory, but that other elements that do not interfere with or contribute to the activity or action of the stated integer or group of integers are optional.

As used in this specification the indefinite articles “a” and “an” may refer to one entity or a plurality of entities and are not to be read or understood as being limited to a single entity.

### DETAILED DESCRIPTION

The present inventors have created an improved formulation, which upon administration to a subject demonstrates anti-inflammatory and/or anti-obesity effects. In this regard, the inventors have surprisingly found that formulations including an edible oil and a flavour oil, such as those essential oils derived from particular fruits, vegetables, herbs and spices, when combined demonstrate synergistic anti-imflammatory effects. The inventors have further found that such formulations may also prove effective in promoting and/or enhancing fat burning and thereby may be effective in decreasing body fat and/or body weight in a subject. To this end, the formulations may demonstrate a variety of therapeutic actions to target obesity including anti-inflammatory actions, appetite suppression and stimulation of lipolysis and fatty acid oxidation.

In one aspect, the invention provides an orally administrable formulation comprising, consisting or consisting essentially of:
(iii) a flavour oil comprising menthol, limonene and/or one or more components or derivatives thereof; and
(iv) an edible oil;
   wherein both the flavour oil and the edible oil have anti-inflammatory properties.

The terms “variant” and “derivative” refer to a modified form of a particular compound or substance. The variant or derivative may be a modified form of a compound or substance that is a component of, for example, the edible oil or flavour oil. Typically, the derivative is a chemically modified or related form of the particular compound or substance.

By “flavour oil” is meant an oil that generally includes volatile compounds having a pleasant aroma, fragrance or smell. Typically, flavour oils or components thereof are obtained from suitable plants, fruit, vegetables, herbs or spices. The flavour oil may be, for example, an essential oil, cold pressed oil, an infused oil or any other lipid-containing extract, fraction or infusion that comprises one or more therapeutically effective elements of the source material from which it is derived. In particular embodiments, the flavour oil is an essential oil.

A flavour oil typically contains the characteristic fragrance of the source material from which it has been derived. It would be apparent to the skilled person that the constituents of a flavour oil may vary depending upon, for example, the production and/or extraction methods used as well as the source material (i.e., fruit, vegetables, plants, herbs, spices etc), from which the oil has been derived. Preferably, the flavour oil of the present invention is suitable for human consumption.

Non-limiting examples of flavour oils include eucalyptus oil, geranium oil, lemongrass oil, petitgrain oil, rosemary oil, thyme oil (white and red), lavender oil, tea tree oil, Tagete minuta oil, lovage oil, Lippia javanica oil, lemon oil, peppermint oil, orange oil, grapefruit oil, oil of bergamot, galbanun oil, pennyroyal oil, pomegranate oil, apple peel oil, cinnamon oil, raspberry oil, strawberry oil, black pepper oil and citrus peel oils. Examples of components or derivatives of flavour oils include, but are not limited to, acetophenone, allyl caprate, α-amylcinnamic aldehyde, amyl salicylate, trans-anethole, anisaldehyde, benzyl alcohol, benzyl acetate, benzyl propionate, bomeol, β-caryophyllene, caryophyllene, cinnamyl acetate, cinnamaldehyde, cinnamic alcohol, cinnamyl alcohol, carvacrol, carveol, citral, citronellal, citronellol, cumin aldehyde, cyclamen aldehyde, decanol, dimethyl salicylate, ethyl butyrate, ethyl caprate, ethyl cinnamate, eucalyptol (cineole), eugenol, iso-eugenol, galaxolide, geranial, geraniol, germacrene D, guaiacol, hexenol, α-hexylcinnamic aldehyde, hydroxycitrolnellal, ionone, ipsdienone, isopropenyl acetophenone, linalol, linalyl acetate, menthol, p-methylacetophenone, methyl anthranilate, methyl dihydrojasmonate, methyl eugenol, methyl ionone, methyl salicylate, neral, α-phellandrene, perillaldehyde, 1- or 2-phenyl ethyl alcohol, 1- or 2-phenyl ethyl propionate, piperine, piperonal, piperitenone, piperonyl acetate, piperonyl alcohol, o-isopropenyl anisole, D-pulegone, terpinen-4-ol, terpinyl acetate, A-tert-butylcyclohexyl acetate, α-terpineol, thymol, trans-tagetenone, myrcenone, linalool, carvone, ipsenone, α-phellandrene, piperitenone, gamma-undecalactone, undecenal, vanillin, and ethyl vanillin.

In one embodiment, the flavour oil is selected from the group consisting of peppermint oil, grapefruit oil, rosemary oil, lemon oil and any combinations thereof.

It would be appreciated that peppermint oil is typically a cold pressed oil, a distilled essential oil and/or an alcohol volatile oil derived from the leaves and flowering tops of Mentha arvense and/or Mentha piperita Linné or a synthetic version thereof. Peppermint oil constituents can vary depending on the source of the plant even though several main constituents are usually present. In general, peppermint oil may contain varying amounts of menthol, menthyl acetate, menthone, piperitone, alpha-pinene, limonene, phellandrene, cadinene, menthyl isovalerate, isovaleric aldehyde, acetaldehyde, menthofuran, cineole, esters of acetic and valeric acids, amyl alcohol, and dimethyl sulfide.

As is well known in the art, grapefruit oil can be derived from the fruit, and in particular the peel or rind thereof, of Citrus Paradisi, Citrus Racemosa or Citrus Maxima, although without limitation thereto. Grapefruit essential oil generally includes limonene, alpha pinene, sabinene, myrcene, geraniol, linalool, citronellal, decyl acetate, neryl acetate, gamma-terpinene, p-cymene and terpinenol.

As is known to one skilled in the art, rosemary oil may be derived from rosemary species such as Rosmarinus officinalis and Rosmarinus coronarium, although without limitation thereto, or a synthetic version thereof. Rosemary oil may include one or more of the following constituents: alpha-terpineol, beta-caryophyllene, borneol, bornyl acetate, bornyl acetate, camphene, camphor, cineole, diosmetin, diosmin, diterpenes, flavonoids including apigenin, genkwanin, hispidulin, isobutyl acetate, limonene, linalool, lutiolin, octanone, phenolic acids (Rosmarinic acid), pinene, saponincineole, sinensetin, terpinen-4-ol, thujone, and/or verbenol.

Lemon oil is typically derived from the rind and skin of the lemon fruit, but may also include synthetic versions thereof. Constituents of lemon oil may include, without limitation thereto, α-pinene, camphene, β-pinene, phellandrene, methyl heptenone, gamma-terpinene, limonene, octaldehyde, citronellol, a-terpineol, citral, linalyl acetate, geraniol, geranyl acetate, nerol, neryl acetate, citronellol, citronellyl acetate, bisabolene, cadinene, methyl anthranilate, limettin and linalool.

As used herein, the term “menthol” means a compound recognized as (1R,2 S, 5R)-2-Isopropyl-5-methylcyclohexanol or 5-methyl-2-(1-methyl ethyl)-cyclohexanol. Menthol may also be referred to as “hexahydrothymol”, “peppermint camphor”, “3-p-menthanol” and “menthomenthol”. The term includes within its scope all isomers of menthol, including the levorotatory (l), dextrorotatory (d) and racemic (dl) forms of 5-methyl-2-(1-methylethyl)-cyclohexanol. Isomers of menthol include (−)-menthol, (+)-menthol, (−)-isomenthol, (+)-isomenthol, (−)-neomenthol, (+)-neomenthol, (−)-neoisomenthol, (+)-neoisomenthol and any mixture thereof. Derivatives of menthol include menthol analogues such as cis-p-menth-2-en-ol, and salts or esters of menthol as a chemical compound and menthol extracts derived from plants of the mint family, for example, the menthol extract of peppermint oil. Further, menthol may be derived from natural sources or synthetically made.

By “limonene” is meant the monocyclic monoterpene 1-methyl-4-(1-methylethenyl)-cyclohexane. The term also includes within its scope all isomers, including its D form (D-limonene equivalent to (+)-limonene or the R enantiomer (R)-(+)-limonene), its L form (L-limonene equivalent to (−)-limonene or the S enantiomer (S)-(−)-limonene), the racemic mixture termed dipentene and any derivatives thereof. Limonene is generally obtained from citrus fruits, and particularly the rind thereof, which typically contain considerable amounts of this compound. To this end, limonene may be derived from natural sources or synthetically made.

The term “edible oil” as used herein, refers to oils, including natural or synthesized oils, which are suitable for human consumption. Preferred edible oils are liquid at 20° C., with many embodiments liquid at 25° C. Edible oils may include vegetable oils, such as soybean, safflower, sunflower, sesame, peanut, corn, olive, rice bran, canola, coconut oil, pomegranate seed oil, palm, cottonseed, rapeseed, carrot, evening primrose, borage, mustard, citrus seed, linseed, chia, avocado, hemp, sugarcane, macadamia, brazil nut, almond, beech nut, hazelnut, cashew, pecan, pine nut, mongongo, pistachio, walnut, pumpkin seed, grapefruit seed, melon seed, gourd seed, blackcurrant, evening primrose, cumin seed, amaranth oil, quinoa oil, apricot seed oil, apple seed, argan, babassu, ben, chestnut, chin nut, carob, cocoa, cocklebur, coriander seed, date seed, dika, grape seed, kapok, kenaf, lallemantia, mefura, niger seed, nutmeg, papaya seed, perilla seed, persimmon seed, pequi, pili nut, poppyseed, pracaxi, prune kernel, ramtil, royle, sacha inchi, sapote, seje, shea, taramira, tes seed, thistle oil, tigernut, tabacco seed, tomato seed, wheat germ, colza, chaulmoogra, candlenut, neem, ojon, passionfruit, rose hip, sea buckthorn, viburnum, tonka bean, ucuhuba seed and meadowfoam. Edible oils may also include animal oils, such as emu oil, ostrich oil, krill oil, cod liver oil, shark liver oil, fish liver oil, halibut liver oil, fish oil, dairy oils, butter, margarines, shortening, lard and ghee. Specific fractions of edible oils may be employed. Additionally, mixtures of oils and/or fractions thereof may be used to perform the present invention.

In one embodiment, the edible oil is selected from the group consisting of fish oil, krill oil, safflower oil, coconut oil, pomegranate seed oil, palm oil, emu oil, and any combination thereof.

Suitably, the edible oil comprises one or more of an omega 3 fatty acid, an omega 6 fatty acid, punicic acid, an omega 7 fatty acid, an omega 9 fatty acid, and any components or derivatives thereof.

In particular embodiments, the omega 3 fatty acid is selected from the group consisting of a-linolenic acid (ALA), hexadecatrienoic acid (HTA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA; clupanodonic acid), docosahexaenoic acid (DHA), tetracosapentaenoic acid, tetracosahexaenoic acid (nisinic acid) and any combination thereof.

In certain embodiments, the omega 6 fatty acid is selected from the group consisting of linoleic acid, γ-linolenic acid (GLA), calendic acid, eicosadienoic acid, dihomo-gamma-linolenic acid (DGLA), docosadienoic acid, adrenic acid, docosapentaenoic acid, tetracosatetraenoic acid, tetracosapentaenoic acid and any combination thereof.

In particular embodiments, the omega 7 fatty acid is selected from the group consisting of palmitoleic acid, vaccenic acid, paullinic acid and any combination thereof.

In certain embodiments, the omega 9 fatty acid is selected from the group consisting of oleic acid, erucic acid, elaidic acid, gondoic acid, mead acid, nervonic acid, and any combination thereof.

As generally used herein, “inflammation” and “inflammatory” refer to the well-known localised response to various types of injury or infection, which is characterised by redness, heat, swelling, and pain, and often also including dysfunction or reduced mobility. Inflammation represents an early defence mechanism to contain an infection and prevent its spread from the initial focus. Major events in inflammation include dilation of capillaries to increase blood flow, changes in the microvasculature structure, leading to escape of plasma and proteins and leukocytes from the circulation, and leukocyte emigration from the capillaries and accumulation at the site of injury or infection.

Non-limiting examples of types of inflammation include acute inflammation, catarrhal inflammation, chronic inflammation, exudative inflammation, fibrinous inflammation, granulomatous inflammation, hyperplastic inflammation, interstitial inflammation, parenchymatous inflammation, hyperplastic inflammation, productive inflammation, proliferative inflammation, pseudomembranous inflammation, purulent inflammation, subacute inflammation, suppurative inflammation and ulcerative inflammation.

Inflammation is often associated with, or secondary to, a disease, disorder and/or condition in a subject, including an immunological disease, disorder and/or condition (such as an autoimmune disease, disorder and/or condition) and an allergic disease, disorder and/or condition. Examples of inflammatory diseases, disorders and/or conditions include, without limitation, Addison's disease, allergic rhinitis, ankylosing spondylitis, asthma, celiac disease, chronic bronchitis, chronic inflammatory demyelinating polyneuropathy (CIDP), chronic obstructive pulmonary disease (COPD), chronic recurrent multifocal ostomyelitis (CRMO), Crohn's disease, ulcerative colitis, demyelinating neuropathies, eczema, emphysema, glomerulonephritis, food allergy, Goodpasture's syndrome, Graves' disease, Guillain-Barre syndrome, Hashimoto's encephalitis, Hashimoto's thyroiditis, hypogammaglobulinemia, idiopathic thrombocytopenic purpura (ITP), insulin-dependent diabetes (typel), juvenile arthritis, Kawasaki syndrome, multiple sclerosis, myasthenia gravis, postmyocardial infarction syndrome, primary biliary cirrhosis, psoriasis, idiopathic pulmonary fibrosis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis.

As used herein, “% concentration” may refer to percent weight/volume (w/v), percent weight/weight (w/w) or percent volume/volume (v/v) of a particular ingredient within the formulation as applicable.

With respect to the formulation of the present aspect, the flavour oil may be present in a % concentration of about 0.5% to about 20% or any range therein such as, but not limited to, about 1% to about 15% or about 2% to about 12%. In particular embodiments the flavour oil is present at a % concentration of about 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20% or any range therein. In particularly preferred embodiments, the flavour oil is present in the formulation at a % concentration of about 2.5% to about 10%.

Further, in reference to the formulation of the present aspect, the edible oil may be present in a % concentration of about 80% to about 99.5% or any range therein such as, but not limited to, about 80% to about 95% or about 85% to about 90%. In particular embodiments the edible oil is present at a % concentration of about 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5% or any range therein. In particularly preferred embodiments, the edible oil is present in the formulation at a % concentration of about 90% to about 97.5%.

Suitably, the formulation comprises about 1% to about 20% peppermint oil and about 80% to about 99% safflower oil. Preferably, the formulation comprises about 5% peppermint oil and about 95% safflower oil.

Suitably, the formulation comprises about 1% to about 20% grapefruit oil, about 0.5% to about 20% peppermint oil, about 10% to about 90% safflower oil, about 10% to about 90% pomegranate seed oil and about 10% to about 90% coconut oil. Preferably, the formulation comprises about 2.5% grapefruit oil, about 7.5% peppermint oil, about 45% safflower oil, about 20% pomegranate seed oil and about 25% coconut oil.

Suitably, the formulation comprises about 0.5% to about 20% grapefruit oil and about 80% to about 99.5% krill oil. Preferably, the formulation comprises about 2.5% grapefruit oil and about 97.5% krill oil.

Suitably, the formulation comprises about 0.5% to about 20% grapefruit oil, about 0.5% to about 20% rosemary oil and about 60% to about 90% fish oil. Preferably, the formulation comprises about 2.5% grapefruit oil, about 2.5% rosemary oil and about 95% fish oil.

Suitably, the formulation comprises about 0.5% to about 20% lemon oil and about 80% to about 99.5% fish oil. Preferably, the formulation comprises about 2.5% lemon oil and about 97.5% fish oil.

Suitably, the formulation comprises about 0.5% to about 20% grapefruit oil and about 80% to about 99.5% fish oil. Preferably, the formulation comprises about 2.5% grapefruit oil and about 97.5% fish oil.

Suitably, the formulation comprises about 0.5% to about 20% grapefruit oil and about 80% to about 99.5% safflower oil. Preferably, the formulation comprises about 2.5% grapefruit oil and about 97.5% safflower oil.

In particular embodiments, the formulation is substantially free of carbohydrate, protein, dietary fibre, alcohol, fillers and/or sweetener.

As is well known to one of skill in the art, a carbohydrate is a biological molecule having carbon (C), hydrogen (H) and oxygen (O) atoms, usually with a hydrogen-oxygen atom ratio of 2:1. Carbohydrates include sugars, starch, and cellulose that are typically divided into the chemical groups of monosaccharides, disaccharides, oligosaccharides, and polysaccharides. The term also includes within its scope carbohydrate derivatives, such as modified starches.

As would be appreciated by the skilled person, a protein is an amino acid polymer. The amino acids may be natural or non-natural amino acids, D- or L-amino acids as are well understood in the art. A protein may be naturally or synthetically derived.

Dietary fibre would be appreciated by the skilled artisan as the indigestible portion of plant-derived food. It typically has two main components, soluble fibre and insoluble fibre, and may include non-starch polysaccharides such as arabinoxylans, cellulose, and other plant components such as resistant starch, resistant dextrins, inulin, lignin, chitins, pectins, beta-glucans, and oligosaccharides.

It would be understood that a filler is an ingredient added to provide bulk or some other non-nutritive purpose to a composition or formulation.

As is well known to one of skill in the art, a sweetener is a sugar substitute that provides a sweet taste like that of sugar while containing significantly less or no food energy. By way of example, the sweetener may include a low calorie sweetener, a natural sweetener, a non-nutritive sweetener and/or an artificial sweetener. Accordingly, the sweetener may be naturally or synthetically derived. Non-limiting examples of sweeteners include stevia, xylitol, aspartame, sucralose, neotame, acesulfame potassium (Ace-K), saccharin, advantame and cyclamates.

By “substantially free” is meant that the orally administrable formulation is either completely free of any carbohydrate, protein, dietary fibre, alcohol, fillers and/or sweetener or it is free to the extent that any carbohydrate, protein, dietary fibre, fillers and/or sweetener which may be present are sufficiently small that their presence does not adversely affect the anti-inflammatory and/or fat burning effects thereof.

Any safe route of administration may be employed for providing a subject with a formulation of the present aspect. For example, oral, rectal, parenteral, sublingual, buccal, intravenous, intra-articular, intra-muscular, intra-dermal, subcutaneous, inhalational, intraocular, intraperitoneal, intracerebroventricular, transdermal, and the like may be employed. Most preferably, the formulation is orally administered.

Dosage forms include powder, tablets, dispersions, suspensions, injections, solutions, syrups, troches, capsules, suppositories, aerosols, transdermal patches, liquid drops, diluted in beverage, gum, confectionary, oral strips, gel, jelly, and the like. These dosage forms may also include injecting or implanting controlled releasing devices designed specifically for this purpose or other forms of implants modified to act additionally in this fashion.

The above formulations may be administered in a manner compatible with the dosage formulation, and in such amount as is pharmaceutically/therapeutically-effective. The dose administered to a subject, in the context of the present invention, should be sufficient to effect a beneficial response (e.g., a reduction in inflammation and/or a reduction in body fat and/or body weight) in a subject over an appropriate period of time. The quantity of the orally administrable formulation to be administered may depend on the subject to be treated, inclusive of the age, sex, weight and general health condition thereof, factors that will depend on the judgement of a practitioner of ordinary skill in the art.

In a further aspect, the invention provides a method of producing the orally administrable formulation according to the aforementioned aspect, including the step of combining the flavour oil comprising menthol, limonene and/or one or more components or derivatives thereof; and the edible oil, wherein both the flavour oil and the edible oil have anti-inflammatory properties, to thereby produce the orally administrable formulation.

The method of the present aspect suitably preserves the anti-inflammatory properties of the flavour oil and the edible oil. By way of example, preserving the anti-inflammatory properties of the flavour oil and/or the edible oil may be achieved by avoiding exposing the formulation to excessive heat. As such, combining the ingredients of the orally administrable formulation at room temperature may improve the efficacy thereof.

In another aspect, the invention provides an orally administrable formulation described herein, or produced according to the method of the aforementioned aspect for use in:

(i) the therapeutic and/or prophylactic treatment of an inflammatory disease, disorder or condition; and/or,

(ii) decreasing and/or preventing an increase in body fat and/or body weight; in a subject.

In a related aspect, the invention provides a method of treating and/or preventing an inflammatory disease, disorder or condition in a subject, said method including the step of administering to said subject a therapeutically effective amount of the orally administrable formulation described herein or produced according to the method hereinbefore described, to thereby treat and/or prevent said inflammatory disease, disorder or condition in the subject.

As used herein, “treating”, “treat” or “treatment” refers to a therapeutic intervention, course of action or protocol that at least ameliorates a symptom of an inflammatory disease, disorder or condition after said disease, disorder or condition and/or its symptoms have at least started to develop. As used herein, “preventing”, “prevent” or “prevention” refers to therapeutic intervention, course of action or protocol initiated prior to the onset of an inflammatory disease, disorder or condition and/or a symptom thereof so as to prevent, inhibit or delay or development or progression of said disease, disorder or condition or the symptom. In this regard, a “prophylactic” treatment is a treatment administered to a subject who does not exhibit signs of an inflammatory disease, disorder or condition or exhibits only early signs for the purpose of decreasing the risk of developing an inflammatory disease, disorder or condition.

By “administration” or “administering” is meant the introduction of an orally administrable formulation (e.g., a formulation comprising, consisting or consisting essentially of a flavour oil and an edible oil, wherein both said oils have anti-inflammatory properties) into a subject by a chosen route, and in particular by the oral route.

The term “therapeutically effective amount” describes a quantity of a specified agent sufficient to achieve a desired effect in a subject being treated with that agent. For example, this can be the amount of an orally administrable a formulation comprising, consisting or consisting essentially of a flavour oil and an edible oil that both demonstrate anti-inflammatory properties to reduce, alleviate and/or prevent an inflammatory disease, disorder or condition. In some embodiments, a “therapeutically effective amount” is sufficient to reduce or eliminate a symptom of such a disease, disorder or condition. In other embodiments, a “therapeutically effective amount” is an amount sufficient to achieve a desired biological effect, for example an amount that is effective to decrease the immune response associated with an inflammatory disease, disorder or condition.

Ideally, a therapeutically effective amount of an agent is an amount sufficient to induce the desired result without causing a substantial cytotoxic effect in the subject. The effective amount of an agent useful for reducing, alleviating and/or preventing an inflammatory disease, disorder or condition will be dependent on the subject being treated, the type and severity of any associated disease, disorder and/or condition, and the manner of administration of the therapeutic composition.

In one embodiment, a given dosage of the orally administrable formulation is applied as a single application or a plurality of applications over a given time period, e.g., for as long as the subject requires treatment, where the dosing schedule is administered over a given time period, examples of which include pre-exercise, intra-exercise and post-exercise, hourly, daily, weekly, biweekly or monthly dosing schedules.

It would also be appreciated that one or more additional agents as are known in the art for reducing, alleviating and/or preventing inflammation (and/or for treating or preventing an inflammatory disease, disorder and/or condition) may be administered to a subject in need thereof in addition to a therapeutically effective amount of the orally administrable formulation described herein. That is, one or more additional agents traditionally used for the treatment and/or prevention of inflammation may be administered to a subject in addition to a therapeutically effective amount of the orally administrable formulation.

For example, nonsteroidal anti-inflammatory drugs (NSAIDs), aminosalicylates, corticosteroids, immunosuppressants, anti-cytokine/cytokine receptor agents (e.g., anti-TNFα agents, anti-IL-5 agents, anti-IL-13 agents, anti-IL-17 agents, and anti-IL-6R agents) particularly anti-cytokine/cytokine receptor antibodies, antibiotics, and combinations thereof can be administered with the orally administrable formulation of the invention in certain embodiments for reducing, alleviating and/or preventing inflammation.

In certain embodiments, the one or more additional agents provide a complimentary or synergistic effect to the action of the orally administrable formulation, preferably eliminating or reducing the frequency or severity of (and/or preventing) one or more symptoms associated with inflammation.

As is well known to one of skill in the art, nonsteroidal anti-inflammatory drugs (NSAIDs), also referred to as nonsteroidal anti-inflammatory agents (NSAIAs), are drugs with analgesic, antipyretic and anti-inflammatory effects, and include salicylates (e.g., aspirin) and propionic acid derivatives (e.g., ibuprofen and naproxen). Aminosalicylates are well known in the art for use in the treatment of inflammatory bowel disease (particularly ulcerative colitis), and include, for example, balsalazide, mesalazine, olsalazine, and sulfasalazine. As is well known to one of skill in the art, corticosteroids are drugs that closely resemble cortisol, a hormone produced by the adrenal glands. Exemplary corticosteroids include, without limitation, cortisone, prednisone, prednisolone, and methylprednisolone. Immunosuppressants are well known in the art for use in the treatment of inflammation associated with certain diseases or conditions, and include, for example, the drugs cyclosporin, azathioprine and mycophenolate. As is well known to one of skill in the art, anti-cytokine/cytokine receptor agents (e.g., anti-TNFα agents, anti-IL-5 agents, anti-IL-13 agents, anti-IL-17 agents, and anti-IL-6R agents) include, without limitation, small molecule inhibitors and antibodies.

Suitably, the inflammatory disease, disorder or condition is selected from the group consisting of Addison's disease, allergic rhinitis, Alzheimer's disease, amyotrophic lateral sclerosis (ALS), ankylosing spondylitis, asthma, atherosclerosis, auto-immunity, cancer-related inflammation, candidiasis, celiac disease, chronic bronchitis, chronic inflammatory demyelinating polyneuropathy (CIDP), chronic obstructive pulmonary disease (COPD), chronic recurrent multifocal osteomyelitis (CRMO), Crohn's disease, ulcerative colitis, dementia, demyelinating neuropathies, eczema, emphysema, glomerulonephritis, food allergy, food intolerance, Good pasture's syndrome, gouty arthritis, Graves' disease, Guillain-Barre syndrome, Hashimoto's encephalitis, Hashimoto's thyroiditis, hypertension, hypercholesterolemia, hypogammaglobulinemia, idiopathic thrombocytopenic purpura (ITP), infection, infestation, inflammatory bowel disease (IBD), insulin resistance, insulin resistance syndrome, insulin-dependent diabetes (type1), intestinal dysbiosis, juvenile arthritis, Kawasaki syndrome, metabolic syndrome, multiple sclerosis, myasthenia gravis, non-alcoholic hepatic steatorrhoea, osteoarthritis, Parkinson's disease, polycystic ovarian syndrome, postmyocardial infarction syndrome, primary biliary cirrhosis, psoriasis, idiopathic pulmonary fibrosis, reactive arthritis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis. In particular preferred embodiments, the inflammatory disease, disorder or condition is selected from the group consisting of asthma, COPD, IBD, ulcerative colitis, osteoarthritis, rheumatoid arthritis, insulin resistance, hypertension, hypercholesterolemia, Alzheimer's disease, amyotrophic lateral sclerosis (ALS), dementia, Parkinson's disease and multiple sclerosis.

In a related aspect, the invention provides a method of decreasing and/or preventing an increase in body fat and/or body weight in a subject, said method including the step of administering to said subject a therapeutically effective amount of the orally administrable formulation hereinbefore described or produced according to the method provided herein, to thereby decrease and/or prevent an increase in body fat and/or body weight in the subject.

It would be apparent that the term “decreasing body fat” refers not only to the effect of decreasing body fat in a subject together with a decrease in body weight as well as the effect of decreasing body fat in a subject despite no change or an increase in body weight. Similarly, the term “decreasing body weight” refers not only to the effect of decreasing body weight in a subject together with a decrease in body fat as well as the effect of decreasing body weight in a subject despite no change or an increase in body fat.

Body fat may be measured directly and/or indirectly by any means known in the art, such as skin fold calipers, biometrical impedance analysis, hydrodensitometry, air displacement (e.g., BodPod®), anthropometric analysis and DEXA scanning, although without limitation thereto. In certain embodiments, decreasing or preventing an increase in body fat includes decreasing and/or preventing an increase in one or more of total body fat, subcutaneous body fat and visceral body fat.

In some embodiments, decreasing body fat of a subject includes reducing body fat, such as total body fat, subcutaneous body fat or visceral body fat, of the subject by at least about 1% (such as at least about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% 25%, or more). In particular embodiments, a reduction in body fat, including total body fat, subcutaneous body fat and visceral body fat, is determined relative to the starting body fat of the subject (for example, prior to treatment with the orally administrable formulation of the invention).

Body weight may be measured directly and/or indirectly by any means known in the art. In particular embodiments, decreasing or preventing an increase in body weight includes decreasing and/or preventing an increase in one or more of total body weight, body mass index (BMI) and waist circumference.

In some embodiments, decreasing body weight of a subject includes reducing body weight, such as that measured by total body weight, BMI or waist circumference, of the subject by at least about 1% (such as at least about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% 25%, or more). In particular embodiments, a reduction in body weight, including that measured by total body weight, BMI or waist circumference, is determined relative to the starting body weight of the subject (for example, prior to treatment with the orally administrable formulation of the invention).

In particular embodiments, the subject is overweight or obese. Obesity is typically defined as a condition that is characterized by excessive accumulation and storage of fat in the body. Individuals suffering from obesity may have various different causative and contributing factors specific to their case; however, typically there are two factors consistently associated with obesity, which are the excessive storage of energy as fat in adipocytes and inflammation.

A subject may be considered overweight or obese if their BMI is greater than 25 kg/m2, their waist circumference is greater than 35 inches (female) or 40 inches (male) or body fat percentage is greater than 25% (male) or 32% (female).

Suitably, the orally administrable formulation may be administered together with a ketogenic diet. As would be appreciated by the skilled artisan, a ketogenic diet is a diet that derives a significant proportion of its energy from fat and only a small proportion of energy from carbohydrates. Such diets are typically designed to force the body to burn fats rather than carbohydrates for energy. Accordingly, following consumption, digestion and metabolism, a ketogenic diet generally provides ketones as a major energy source. These ketonic compounds include, for example, aceto acetate, D-3 hydroxy butyrate and acetone. A ketogenic diet may induce a metabolic state known as ketosis, characterised by elevated levels of circulating ketone bodies (e.g., serum ketone body levels over 0.5 millimolar), together with low and stable levels of insulin and blood glucose. In particular embodiments, the orally administrable formulation promotes, enhances and/or prolongs ketosis in the subject.

With respect to the method of the present aspect, it would be readily apparent that the orally administrable formulation may also be administered before, during and/or after an exercise so as to further enhance or promote its fat burning properties. To this end, the exercise may be any broadly known in the art, including aerobic exercise (e.g., cardiovascular exercise) and anaerobic exercise (e.g., HIIT and resistance exercise).

The term “subject”, as used herein, includes both human and veterinary subjects. For example, administration to a subject can include administration to a human subject or a veterinary subject. Preferably, the subject is a human. However, therapeutic uses according to the invention may also be applicable to mammals such as domestic and companion animals, performance animals such as horses, livestock, and laboratory animals.

All computer programs, algorithms, patent and scientific literature referred to herein is incorporated herein by reference.

So that the present invention may be more readily understood and put into practical effect, the skilled person is referred to the following non-limiting examples.

### EXAMPLE 1

A formulation consisting of grapefruit oil (2.5%) and peppermint oil (7.5%) combined with safflower oil (45%), pomegranate seed oil (20%) and coconut oil (25%) was tested in a preclinical trial for obesity.

### Method

2 ml of the liquid oil formulation was consumed orally by test subjects both before and during exercise. Subjects were interviewed immediately upon consumption and again post-workout and answers were recorded.

### Results

TABLE 1

| | | | Perceived improvement in | |
|---|---|---|---|---|
| | | | workout/10 (with 10 being | |
| | | Digestibility/10 (1 being | best workout ever and 5 | |
| | | evacuation/purgative and | being average and 0 being | Generation |
| Subject | Taste/10 | 10 being perfectly fine) | worst workout ever) | of ketones |
| 1 | 6/10 | 8/10 | 7/10 | Yes during fasted |
| | | | | cardio and resistance |
| | | | | training |
| 2 | 7/10 | 8/10 | 8/10 | No (not fasting |
| | | | | in bulking phase) |
| 3 | 5/10 | 5/10 | 9/10 | NA |
| 4 | 5/10 | 7/10 | 6/10 | NA |
| 5 | 7/10 | 5/10 | 7/10 | Yes with |
| | | | | ketogenic diet |
| 6 | 6/10 | 5/10 | 8/10 | NA |
| 7 | 5/10 | 5/10 | NA | NA |
| 8 | 5/10 | 3/10 | 5/10 | NA |
| 9 | 7/10 | 5/10 | 9/10 | NA |

### Further Comments:

The mouth feel was well tolerated with and without emulsifying agent in 100% of subjects. One subject didn't like it but tolerated it and continued to use the product and has requested more. This subject explained they had “acquired a taste”.
Essential oil for flavouring was well tolerated in 100% of subjects.
Subjects report increased arousal and perceived energy.
Subjects anecdotally reported improved exercise performance.
When combined with a ketogenic diet, the formulation produced an enhanced and prolonged ketosis in subjects.
When used prior to a fasted cardio exercise, the formulation enhances exercise performance and ketone body generation.
When used prior to cardiovascular exercise and resistance exercise, the formulation showed enhanced exercise performance
Other feedback
   Reduced appetite
   Enhanced mood
   Increased mental clarity and focus
   Improved perceived cognition
   Improved vision and focus
   Reduced joint pain
   Reduced DOMS (delayed onset muscle soreness)
   Improved gut function and bowel frequency
   Reduction in need for bronchodilator during exercise

1. An orally administrable formulation comprising:
(i) a flavour oil comprising an oil selected from the group consisting of menthol oil, limonene oil, peppermint oil, grapefruit oil, rosemary oil, lemon oil, and any combination thereof and/or one or more components or derivatives thereof; and
(ii)an edible oil;
wherein both the flavour oil and the edible oil have anti-inflammatory properties.

2. The orally administrable formulation of claim 1, wherein the flavour oil is selected from the group consisting of peppermint oil, grapefruit oil, rosemary oil, lemon oil and any combination thereof.

3. The orally administrable formulation of claim 1, wherein the edible oil comprises one or more of an omega 3 fatty acid, an omega 6 fatty acid, punicic acid, an omega 7 fatty acid, an omega 9 fatty acid, and any compnnents nr derivatives thereof.

4. The orally administrable formulation of claim 3, wherein the omega 3 fatty acid is selected from the group consisting of ct-linolenic acid (ALA), hexadecatrienoic acid (HTA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA; clupanodonic acid), docosahexaenoic acid (DHA), tetracosapentaenoic acid, tetracosahexaenoic acid (nisinic acid) and any combination thereof.

5. The orally administrable formulation of claim 3, wherein the omega 6 fatty acid is selected from the group consisting of linoleic acid, γ-linolenic acid (GLA), calendic acid, eicosadienoic acid, dihomo-gamma-linolenic acid (DGLA), docosadienoic acid, adrenic acid, docosapentaenoic acid, tetracosatetraenoic acid, tetracosapentaenoic acid and any combination thereof.

6. The orally administrable formulation of claim 3, wherein the omega 7 fatty acid is selected from the group consisting of palmitoleic acid, vaccenic acid, paullinic acid and any combination thereof.

7. The orally administrable formulation of claim 3, wherein the omega 9 fatty acid is selected from the group consisting of oleic acid, erucic acid, elaidic acid, gondoic acid, mead acid, nervonic acid, and any combination thereof.

8. The orally administrable formulation of claim 1, wherein the edible oil is selected from the group consisting of fish oil, krill oil, safflower oil, coconut oil, pomegranate seed oil, palm oil, emu oil and any combination thereof.

9. The orally administrable formulation of claim 1, wherein the formulation comprises from about 0.5% to about 20% of the flavour oil and from about 80% to about 99.5% of the edible oil.

10. The orally administrable formulation of claim 9, wherein the formulation comprises from about 2.5% to about 10% of the flavour oil and from about 90% to about 97.5% of the edible oil.

11. The orally administrable formulation of claim 1, wherein the formulation comprises about 1% to about 20% peppermint oil and about 80% to about 99% safflower oil.

12. The orally administrable formulation of claim 11, wherein the formulation comprises about 5% peppermint oil and about 95% safflower oil.

13. The orally administrable formulation of claim 1, wherein the formulation comprises about 1% to about 20% grapefruit oil, about 0.5% to about 20% peppermint oil, about 10% to about 90% safflower oil, about 10% to about 90% pomegranate seed oil and about 10% to about 90% coconut oil.

14. The orally administrable formulation of claim 13, wherein the formulation comprises about 2.5% grapefruit oil, about 7.5% peppermint oil, about 45% safflower oil, about 20% pomegranate seed oil and about 25% coconut oil.

15. The orally administrable formulation of claim 1, wherein the formulation comprises about 0.5% to about 20% grapefruit oil and about 80% to about 99.5% krill oil.

16. The orally administrable formulation of claim 15, wherein the formulation comprises about 2.5% grapefruit oil and about 97.5% krill oil.

17. The orally administrable formulation of claim 1, wherein the formulation comprises about 0.5% to about 20% grapefruit oil, about 0.5% to about 20% rosemary oil and about 60% to about 90% fish oil.

18. The orally administrable formulation of claim 17, wherein the formulation comprises about 2.5% grapefruit oil, about 2.5% rosemary oil and about 95% fish oil.

19. The orally administrable formulation of claim 1, wherein the formulation comprises about 0.5% to about 20% lemon oil and about 80% to about 99.5% fish oil.

20. The orally administrable formulation of claim 19, wherein the formulation comprises about 2.5% lemon oil and about 97.5% fish oil.

21. The orally administrable formulation of claim 1, wherein the formulation comprises about 0.5% to about 20% grapefruit oil and about 80% to about 99.5% fish oil.

22. The orally administrable formulation of claim 21, wherein the formulation comprises about 2.5% grapefruit oil and about 97.5% fish oil.

23. The orally administrable formulation of claim 1, wherein the formulation comprises about 0.5% to about 20% grapefruit oil and about 80% to about 99.5% safflower oil.

24. The orally administrable formulation of claim 23, wherein the formulation comprises about 2.5% grapefruit oil and about 97.5% safflower oil.

25. The orally administrable formulation of claim 1, wherein the formulation is substantially free of carbohydrate, protein, dietary fibre, alcohol, fillers and/or sweetener.

26. A method of producing the orally administrable formulation according to claim 1, including combining the flavour oil and the edible oil, wherein both the flavour oil and the edible oil have anti-inflammatory properties, to thereby produce the orally administrable formulation.

27. (canceled)

28. A method of treating and/or preventing an inflammatory disease, disorder or condition in a subject, said method including administering to said subject a therapeutically effective amount of the orally administrable formulation of claim 1.

29. The method of claim 28, wherein the inflammatory disease, disorder or condition is selected from the group consisting of Addison's disease, allergic rhinitis, Alzheimer's disease, amyotrophic lateral sclerosis (ALS), ankylosing spondylitis, asthma, atherosclerosis, auto-immunity, cancer-related inflammation, candidiasis, celiac disease, chronic bronchitis, chronic inflammatory demyelinating polyneuropathy (CIDP), chronic obstructive pulmonary disease (COPD), chronic recurrent multifocal osteomyelitis (CRMO), Crohn's disease, ulcerative colitis, dementia, demyelinating neuropathies, eczema, emphysema, glomerulonephritis, food allergy, food intolerance, Good pasture's syndrome, gouty arthritis, Graves' disease, Guillain-Barre syndrome, Hashimoto's encephalitis, Hashimoto's thyroiditis, hypertension, hypercholesterolemia, hypogammaglobulinemia, idiopathic thrombocytopenic purpura (ITP), infection, infestation, inflammatory bowel disease (IBD), insulin resistance, insulin resistance syndrome, insulin-dependent diabetes (type1), intestinal dysbiosis, juvenile arthritis, Kawasaki syndrome, metabolic syndrome, multiple sclerosis, myasthenia gravis, non-alcoholic hepatic steatorrhoea, osteoarthritis, Parkinson's disease, polycystic ovarian syndrome, postmyocardial infarction syndrome, primary biliary cirrhosis, psoriasis, idiopathic pulmonary fibrosis, reactive arthritis, Reiter's syndrome, rheumatoid arthritis, sarcoidosis, scleroderma, Sjogren's syndrome, systemic lupus erythematosus (SLE), thrombocytopenic purpura (TTP), ulcerative colitis, vasculitis, vitiligo, and Wegener's granulomatosis and any combination thereof.

30. A method of comprising administering to a subject a therapeutically effective amount of the orally administrable formulation according to claim 1.

31. The method of claim 30, wherein the orally administrable formulation is administered to the subject together with a ketogenic diet.

32. The method of claim 30, wherein the orally administrable formulation promotes, enhances and/or prolongs ketosis in the subject.

33. The method of claim 30, wherein the orally administrable formulation is administered before, during and/or after an exercise.

34. (canceled)
